(19) United States
(12) Patent Application Publication
(10) Pub. No.: US 2008/0188564 A1
(43) Pub. Date: Aug. 7, 2008

(54) Insecticidal and Nematicidal Compositions and Methods of Use

(76) Inventors: Jeffrey R. Bloomquist, Blacksburg, VA (US); Edwin E. Lewis, Davis, CA (US)

Correspondence Address: LATIMER, MAYBERRY & MATTHEWS IP LAW, LLP, 13873 PARK CENTER ROAD, SUITE 106, HERNDON, VA, 20171, US

(21) Appl. No.: 11/720,117

(22) PCT Filed: Nov. 29, 2005
(86) PCT No.: PCT/US05/42977

§ 371 Date: Aug. 16, 2007

Related U.S. Application Data
(60) Provisional application No. 60/631,579, filed on Nov. 30, 2004.
(60) Provisional application No. 60/730,386, filed on Oct. 27, 2005.

(51) Int. Cl. A61K 31/185; G01N 33/50; G01N 25/18; A61P 33/00

(52) U.S. Cl. 514/576; 436/501; 436/149

(57) ABSTRACT

The present invention provides methods of protecting plants and other multicellular organisms from the parasitic activity of nematodes and insects. The methods use compounds, such as stilbene compounds, as nematicidal and insecticidal compounds, which act to alter the activity of one or more anion transporters in the nematodes and insects. Methods of screening for such compounds are also provided.

### CROSS-REFERENCE TO RELATED APPLICATIONS

This application relies on the disclosure of and claims the benefit of the filing date of U.S. provisional patent application No. 60/631,579, filed on 30 Nov. 2004, and U.S. provisional patent application No. 60/703,386, filed 27 Oct. 2005, the entire disclosures of both of which are hereby incorporated herein by reference.

### BACKGROUND OF THE INVENTION

1. Field of the Invention

The present invention relates to compounds, compositions, and methods for inhibiting the growth of, or for killing, insects and nematodes. It also relates to methods of identifying compounds having insecticidal or nematicidal activity. More specifically, the invention provides stilbene compounds having activity against anion transporters, use of those compounds as insecticides and nematicides, and use of anion transporters to identify compounds having insecticidal and/or nematicidal activity.

2. Description of Related Art

Nematodes and insects are economically important pests in the agriculture and animal husbandry industries. Their combined yearly damage in these two fields in the U.S. alone amounts to well in excess of tens of millions of dollars. In view of this recurring annual economic loss, much research has been performed to identify compounds that have insecticidal or nematicidal activity. Various insecticidal or nematicidal compounds, ranging from simple chemical compounds produced through chemical syntheses (e.g., carbamates) to toxins produced by bacteria or other living organisms to transgenic crops that are resistant to attacks, have been developed or proposed. However, because insects and nematodes are living organisms subject to evolution through selective pressure, resistance to insecticides and nematicides is seen. Therefore, there is a continual need for novel insecticides and nematicides.

Nematodes are tiny, worm-like, multicellular organisms found in virtually all habitats throughout the world, including water and soil. While the number of nematode species has been estimated in the hundreds of thousands, only a small portion of those are parasitic and considered as pests. Plant parasitic nematodes are found associated with most agriculturally important plants. While many are plant species-specific in their targeting, most are promiscuous, capable of parasitizing two or more plant species. The major pathway of infection for soil-dwelling plant-parasitic nematodes is through the root tissue. Their damage to the roots can diminish the plant's ability to take up water and nutrients, and can provide portals of entry for other plant diseases.

In general, protection of plants from nematodes is focused on blocking attack of nematodes in the first place. Typically, the soil, water, or plants are treated with a nematicide prior to or at the time of sowing seeds, at the time of seedling germination, or during growth of the plants.

Crop and other plant damage from insects typically results from the activity of the insects at the larval and adult stages. Larvae generally cause damage by feeding on foliage, shoots, and roots. This damage can be so severe, that a significant portion of a particular crop can be lost if no insecticides are applied. The most noticeable damage caused by adult insects is damage caused by eating foliage and fruits of plants, but equally severe damage is also caused through eating of stems and roots or by sucking liquids from stems, fruits, and foliage. Again, significant damage to crops or other plants can occur if protective or remedial action is not timely taken.

At the molecular level, studies have focused on determining the mechanisms of action of various insecticides and nematicides, often with the goal of identifying the cellular targets for certain active compounds. Of the many cellular targets, cell-surface transporters or receptors are important because of the number present and the relatedness of many across species (i.e., a particular compound that acts on one might be effective in treating numerous species).

It is known to use certain stilbene compounds to treat or protect plants and animals from attack by nematodes and insects. For example, U.S. Pat. No. 4,271,186 to Foerster et al. discloses stilbene derivatives having insecticidal and acaricidal activity. The compounds are disclosed as being useful for treating both plants and animals. In addition, U.S. Pat. No. 5,246,936 to Treacy et al. discloses the use of a combinations of pesticides and stilbene compounds to enhance the activity of the pesticide. See, for example, the Abstract of the '936 patent.

Furthermore, U.S. Pat. No. 5,314,693 to Suga et al. discloses hydroxystilbenes and salts thereof, which are said to have nematicidal activity against pine wood nematodes. See, for example, the Abstract and Summary of the Invention of the '693 patent. In addition, U.S. Pat. No. 5,530,030 to Suga et al. discloses the use of chlorinated hydroxystilbenes or salts thereof as nematicides against the pine wood nematode. See, for example, the Abstract and Summary of the Invention of the '030 patent.

The use of stilbene compounds in conjunction with other killing agents has also been disclosed. For example, U.S. Pat. No. 5,662,897 to Miller et al. discloses the use of a combination of a baculovirus and a stilbene compound to infect and kill insects. More specifically, the '897 patent discloses an engineered insect-killing virus. The patent further discloses that the killing effectiveness of the virus can be enhanced by co-treatment of the insect with the virus and a stilbene compound. See, for example, the '897 patent at column 5, lines 65-67, and column 23, lines 41-63.

There are also examples of insecticides usually classified as gamma-aminobutyric acid (GABA) antagonists or sodium channel agonists that interact with anion transporters (ATs) (e.g., voltage-dependent chloride channels), which were reviewed by Bloomquist (2003). For example, Bloomquist, J. R. (“Intrinsic lethality of chloride-channel-directed insecticides and convulsants in mammals” Toxicol. Lett. 60:289-298, 1992) discloses comparative studies of intraperitoneal versus intracerebral injection in mice. The studies found little or no potentiation of toxicity after intracerebral injection of lindane, abamectin, TBPS, and p-CN-TBOB, all compounds that act on GABA receptors and which should have potentiated toxicity when injected into the brain. The lack of potentiation supported the possibility of effects on other, perhaps peripheral sites, such as voltage-sensitive chloride channels. No physiological data were provided, and this study was done in mice.

In addition, Abalis, I., et al. (“Binding of GABA receptor channel drugs to a putative voltage-dependent chloride channel in Torpedo electric organ”, Biochem. Pharmacol. 34:2579-2582, 1985) suggests the involvement of voltage-gated chloride channels in the mode of action of these compounds. [³⁵S]TBPS binding studies were performed using the Torpedo nobiliana (fish) electric organ, which lacks GABA receptors, but does possess voltage-gated chloride channels. In this tissue, the binding displacement by picrotoxinin and endrin were of relatively low potency compared to displacing binding in rat brain membranes. In contrast, lindane was about 4-fold more potent as an inhibitor of binding to Torpedo membranes than rat brain, and was a more effective inhibitor than either picrotoxinin or endrin. No toxicity data were provided, and this study was done in fish.

Payne, G. T. and Soderlund, D. M. (“Activation of γ-aminobutyric acid insensitive chloride channels in mouse brain synaptic vesicles by avermectin B1a”, J. Biochem. Toxicol. 6:283-292, 1991) and Payne G. T. and Soderlund, D. M. (“Actions of avermectins on γ-aminobutyric acid (GABA)-sensitive and GABA-insensitive chloride channels in mouse brain”, Pestic. Biochem. Physiol. 47:178-184, 1993) show that avermectins interact with voltage-dependent chloride channels of mouse brain. Abamectin stimulated radiochloride efflux from mammalian brain vesicular preparations that are sensitive to block by DIDS, an established blocker of voltage-dependent chloride channels (Payne and Soderlund, 1991), and structure-activity studies found that seven abamectin analogs stimulated efflux with half maximal potencies of around 1 micromolar (uM) (Payne and Soderlund, 1993). From this work, the authors concluded that interaction with GABA-insensitive chloride channels may contribute to the mammalian neurotoxicity of the avermectins.

Ray, D. E., et al. (“Action of pyrethroid insecticides on voltage-gated chloride channels in neuroblastoma cells”, Neurotoxicology 18:755-760, 1997) and Forshaw, P. J., et al. (“The role of voltage-gated chloride channels in type II pyrethroid insecticide poisoning”, Toxicol. Appl. Pharmacol. 163:1-8, 2000) disclose patch clamp studies of neuroblastoma. The authors found a class of chloride channels that was sensitive to blockage by type 2 pyrethroids (Ray et al., 1997). Neuroprotection studies with ivermectin and barbiturates suggested that the effects of ivermectin and phenobarbital on intoxication by deltamethrin were specifically related to an action on voltage-sensitive chloride channels (Forshaw et al., 2000). Both of these papers studied the neurotoxicity of pyrethroids in mammalian tissue/animals.

Machaca, K., et al. (“A novel chloride channel localizes to Caenorhabditis elegans spermatids and chloride channel blockers induce spermatid differentiation”, Dev. Biol. 176(1):1-16, 1996) discloses use of AT blockers to characterize AT-mediated functions. As a part of the work reported in this paper, the authors found that trans-4,4′-diisothiocyanatostilbene-2,2′-disulfonic acid (DIDS) treatment induced differentiation of spermatozoa in the free living nematode Caenorhabditis elegans. This nematode is a model organism for many types of studies quite apart from any consideration of nematicidal action (Bazzicalupo, 1983), and spermatid differentiation has no association with acute toxicity.

Scott-ward, T. S., et al. (“Direct block of the cystic fibrosis transmembrane conductance regulator Cl(−) channel by niflumic acid”, Mol. Membr. Biol. 21(1):27-38, 2004) discloses that a number of AT channel blockers have been investigated as possible drug candidate molecules, especially for treatment of cystic fibrosis, a disease where cellular chloride ion regulation is deranged. In this study, niflumic acid was used to block the cystic fibrosis transmembrane conductance regulator (CFTR) Cl(−) channel. The authors conclude that niflumic acid is an open-channel blocker of CFTR that inhibits Cl(−) permeation by plugging the channel pore. This effect is similar to that of DIDS on the AT. The authors go on to state that niflumic acid or related agents might be of value in the development of new therapies for autosomal dominant polycystic kidney disease, much as the anion transporter inhibitor furosemide is used as a diuretic (Cabantchik and Greger, 1992).

As can be seen, the state of the art is such that stilbene compounds are known to be useful as nematicides and insecticides. Blockers of AT have also been studied as probes of channel function and as drug candidates in mammals. AT blockage has been identified as an ancillary effect of insecticidal compounds known to work primarily on ligand-gated chloride channels. However, it has not been shown or suggested in the art that any compound that is active against an AT has any in vivo nematicidal activity. Likewise, it has not been shown or suggested that any compound that has in vivo nematicidal activity affects an AT. In addition, it has not been shown or suggested in the art that any compound that is active against an AT has any in vivo insecticidal activity. By the same token, it has not been shown or suggested that any compound that has in vivo insecticidal activity affects an AT.

### SUMMARY OF THE INVENTION

The present invention provides compounds, and compositions comprising those compounds, having anti-nematode activity (e.g., nematicidal, protozoacidal), insecticidal activity, and acaricidal activity. Exemplary embodiments relate to stilbene compounds. It also provides methods of treating plants and other multicellular organisms (e.g., fish, livestock, companion animals, humans) with compounds, such as stilbene compounds, and compositions comprising them to protect the treated organism from attack by insects and nematodes, to treat them for infestation or the parasitic activity of insects and nematodes, or to rid them of insects and nematodes attacking or parasitizing them. In addition, the invention provides methods of identifying compounds, including stilbene compounds, that show insecticidal, nematicidal, or acaricidal activity against one or more insects, nematodes, or Acari (e.g., ticks, mites). The methods use one or more anion transporters (AT) as screening agents to identify compounds that bind to and block the activity of the AT. As used herein, AT are anion exchangers, anion co-transporters, and voltage-sensitive ion channels.

In a first aspect, the invention provides stilbene compounds having a core structure that is described as a trans-α,β-diphenyletheylene, and as depicted in FIG. 1A, and compositions containing such stilbene compounds. The compounds of the invention can be substituted about one or more carbons of one or both of the phenyl rings of the stilbene core structure. The invention further provides other compounds, which do not have a stilbene core structure. Regardless of the structure, the compounds provided by the invention have anti-nematode, anti-insect, and/or anti-Acari activity as a result of their effect on one or more AT of these organisms.

In a second aspect, the present invention provides compositions comprising stilbene compounds. The compositions can be used for treatment of plants, animals, or the environment surrounding selected plants and/or animals. Treatment can be for inhibiting the growth or reproduction of one or more insects or nematodes present in, on, or around the plants, animals, or environment. In embodiments, treatment is for killing of one or more insects or nematodes. In general, the compositions comprise one or more compounds having nematicidal, insecticidal, and/or acaricidal activity, such as stilbene compounds, and another substance, such as a compatible carrier or binder, another biologically active agent, a dispersant, a solvent, or the like. In preferred embodiments, the compositions comprise one or more substance that improves the solubility of a stilbene in water or an aqueous liquid.

In a third aspect, the present invention provides methods of treating plants, animals (including humans), and their environments that are parasitized, infected, damaged, or killed by one or more insect, nematode, and/or Acari species, including, but not limited to free-living and plant parasitic nematodes. It also provides methods of treating plants and animals that are bitten, damaged, or killed by one or more insect, Acari and/or nematode species. Inhibition can be by reducing the viability of the nematode, insect, or Acari organisms, resulting in ultimate death of the organisms, or by direct killing of the organisms. The methods generally comprise exposing or contacting the plants, animals, or environments to at least one compound, such as a stilbene compound, in a sufficient amount to inhibit the activity or viability of at least one nematode, insect, or Acari, or a sufficient amount to directly kill at least one nematode, insect, or Acari. Stated another way, the methods generally comprise exposing or contacting the plants, animals, or environments to at least one compound in a sufficient amount to inhibit the activity or viability of at least one nematode, insect, or Acari, or a sufficient amount to directly kill at least one nematode, insect, or Acari, where the compound is identified through a method of identification or screening according to the present invention.

In a fourth aspect, the present invention provides methods of treating nematodes, insects, or Acari that parasitize, damage, or kill plants or animals, including but not limited to humans, to inhibit them from parasitizing, damaging, or killing the plants or animals. Inhibition can be by reducing the viability of the nematode, insect, or Acari organisms, resulting in ultimate death of the organisms, or by direct killing of the organisms. The methods generally comprise exposing or contacting the nematodes, insects, or Acari to at least one compound, such as a stilbene compound, in a sufficient amount to inhibit the activity or viability of at least one nematode, insect, or Acari or a sufficient amount to directly kill at least one nematode, insect, or Acari. Stated another way, the methods generally comprise exposing or contacting the nematodes, insects, or Acari to at least one compound in a sufficient amount to inhibit the activity or viability of at least one nematode, insect, or Acari, or a sufficient amount to directly kill at least one nematode, insect, or Acari where the compound is identified through a method of identification or screening according to the present invention.

In a fifth aspect, the invention provides methods of identifying compounds, such as stilbene compounds, that show insecticidal, nematicidal, or acaricidal activity against one or more insects, nematodes, or Acari. The methods use one or more anion transporters (AT) as screening agents to identify compounds that bind to and block the activity of the AT. The methods generally comprise contacting one or more AT molecules with one or more compounds and determining if the activity of the AT was altered, alteration of activity indicating that one or more of the compounds affected the activity of the AT. While it is preferred that the activity of the AT be inhibited, diminished, etc., the methods of the invention may also identify compounds that activate, enhance, etc. the activity of one or more AT. Such activators may be used for various purposes, including, but not limited to, use as competitors for characterization of certain AT inhibitors, and use as anti-nematode compounds, as insecticides, or as acaricides (disruption of AT activity, whether inhibition or over-activation should disrupt normal cellular activity and result in loss in viability and/or death). Although the methods can be practiced in vivo, typically they are performed in vitro, or initially performed in vitro, with confirmatory assays performed in vivo.

### BRIEF DESCRIPTION OF THE DRAWINGS

The accompanying drawings, which are incorporated in and constitute a part of this specification, illustrate several embodiments of the invention, and together with the written description, serve to explain various principles of the invention. It is to be understood that the drawings do not limit the scope of the invention, but are provided merely to aid in understanding of certain aspects and embodiments of the invention.

FIG. 1A depicts the chemical structure of stilbene (trans-α,β-diphenyletheylene).

FIG. 1B depicts the chemical structure of 3,5-dihydroxy-4-isopropylstilbene (DST).

FIG. 1C depicts the chemical structure of trans-4,4′-diisothiocyanatostilbene-2,2′9disulfonic acid (DIDS).

FIG. 2 depicts the results of a toxicity bioassay of DIDS to nematodes.

### DETAILED DESCRIPTION OF VARIOUS EMBODIMENTS OF THE INVENTION

Reference will now be made in detail to various exemplary embodiments of the invention. It is to be understood that the following detailed description is presented for the purpose of describing certain embodiments in detail. Thus, the following detailed description is not to be considered as limiting the invention to the embodiments described. Rather, the true scope of the invention is defined by the claims.

As used herein, the term “nematode” and its various forms are used as a short-hand for all organisms of the phylum Nematoda, including, but not necessarily limited to, all organisms in the various classes that have been traditionally used, are in current use, or are proposed for use in the future. The term thus includes organisms classically referred to as Metazoan organisms. It thus encompasses all organisms in the classes Adenophorea and Secernentea, but is not limited only to those organisms. The use herein of the term nematode is to be understood to encompass all such organisms, without limitation to current or prior taxonomic or phylogenic schemes or labels. In preferred embodiments, the term refers to parasitic nematodes, such as those that infect animals (including humans) and those that infect plants. In other embodiments, the term refers to free-living nematodes.

The term “insect” and its various forms is used herein to indicate all organisms within the class Insecta. The term thus includes, but is not necessarily limited to, organisms that are currently classified as members of the class Insecta. It also includes, but is not necessarily limited to, organisms that were previously considered to be members of the class or have been traditionally considered insects. Likewise, the term “Acari” and all of its forms (e.g., acarine) is used to indicate all organisms that are currently classified as members of the order Acari. The term thus includes, but is not limited to, mites and ticks.

The present invention is based, at least in part, on the discovery that the transporter activity of anion transporters (AT) is critical for viability of nematodes, insects, and Acari and thus can be used as a target for killing of these agriculturally important pests. It has now been discovered that the activity of AT can be inhibited by certain compounds, such as stilbene compounds, and that such inhibition is inhibitory to the growth and activity of nematodes, insects, and Acari, either when the compounds are applied to the organisms or to the plants or animals they attack (or the environment surrounding the plants or animals). Based on this discovery, it is now recognized that certain compounds, such as certain stilbene compounds, can be specifically identified and used to kill specific nematodes, insects, and Acari. In essence, the present invention provides a powerful mode of action for pesticidal activity of compounds, including stilbenes, in nematodes, insects, and Acari and opens numerous therapeutic, agricultural, and research fields for treatment of animals, plants, and the environment, and for discovery of new compounds for treatment of animals plants, and the environment.

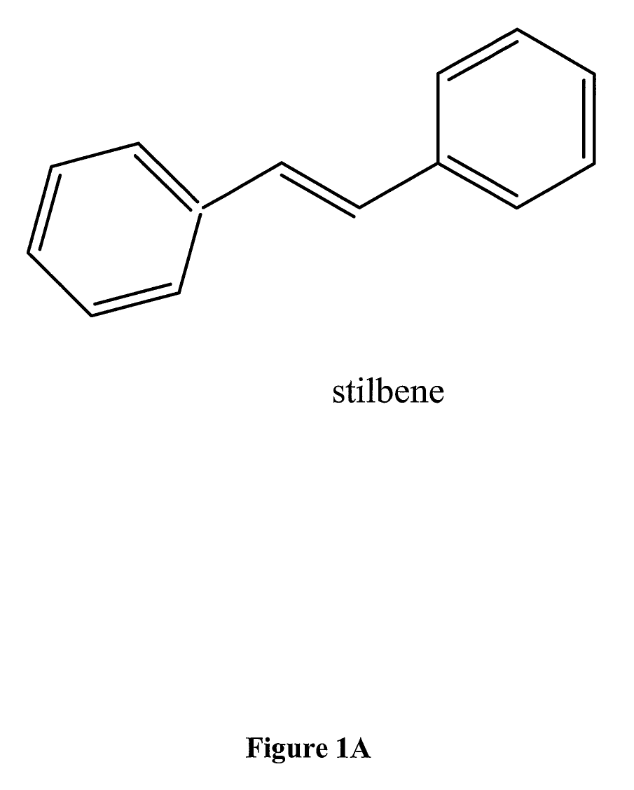

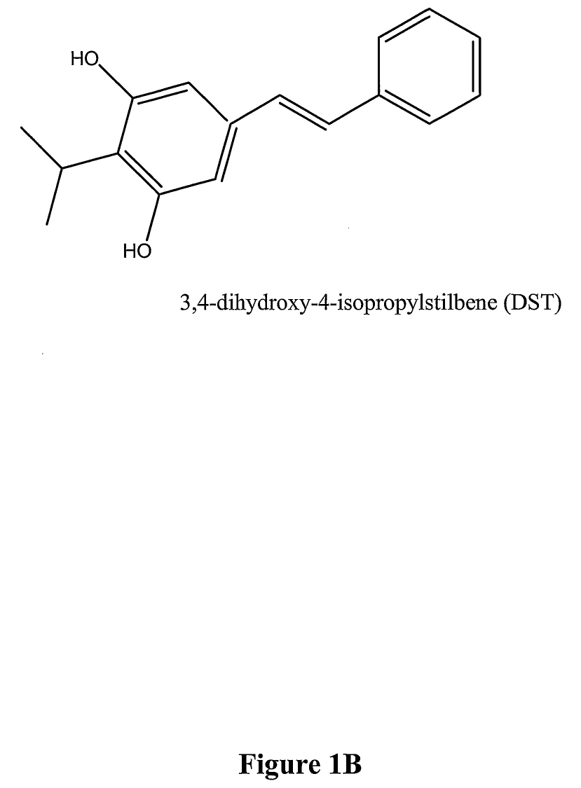

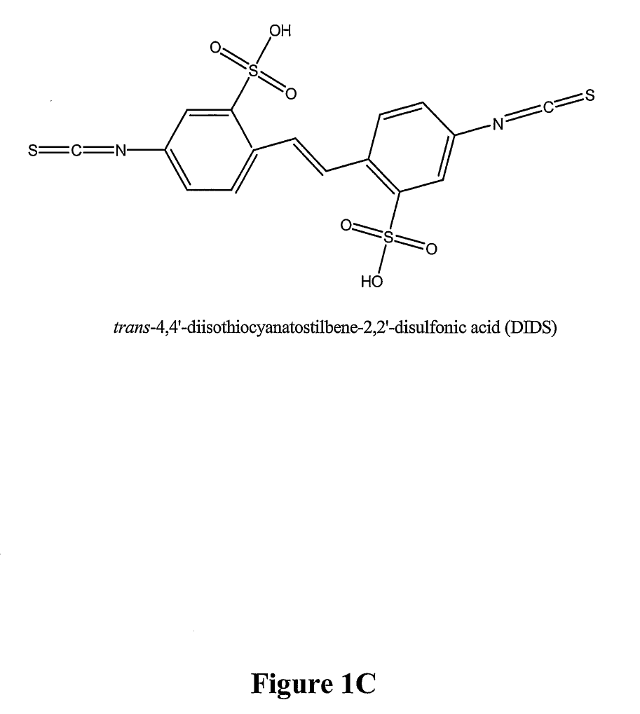

In a first aspect, the invention provides stilbene compounds having a core structure that is described as a trans-α,β-diphenyletheylene, and as depicted in FIG. 1A. As can be seen from FIG. 1A, the core stilbene structure comprises two phenyl groups linked via an ethylene bridge. According to the invention, stilbene compounds can be any compound having this core structure. Thus, stilbene compounds according to the invention can be substituted at one or more carbons of one or both of the phenyl groups. When more than one carbon is substituted with an atom or group, each substituent can be the same, two or more (but not all) can be the same, or each can be different from all others.

The carbons of the phenyl groups can be substituted with any element or group known in the art as suitable for bonding to phenyl carbons. Examples include, but are not limited to, elements or protonated forms of elements, such as hydrogen, carbon, nitrogen, oxygen, and sulfur; organic groups, such as short-chain (1-4 carbon), medium chain (5-12 carbon), and long-chain (13 or greater) carbonyl groups, such as substituted or unsubstituted alkane, alkene, alkyl, alkylene, and alkenyl groups; substituted and unsubstituted aryl groups, such as phenyl groups; nitrogen-containing groups; sulfur-containing groups; metal-containing groups, halide-containing groups, and the like. In addition, one or more of the substituent groups may be substituted in accordance with the listing presented for the phenyl groups of the stilbene core structure. Furthermore, either or both of the carbons of the ethylene linkage may be substituted with one or more of the groups listed above.

In general, the stilbene compounds of the invention can have the following formula:

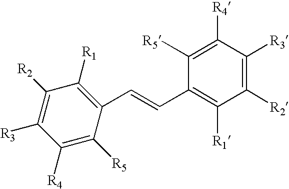

in which all of R₁-R₅ and R₁′-R₅′ are identical or independently selected from: H; OH; N or a substituted nitrogenous group, for example, NR₇R₈, wherein R₇ and R₈ are independently hydrogen, substituted or unsubstituted alkyl or aryl, or R₇ and R₈ combine with the joining nitrogen atom to form a heterocyclic radical, such as a morpholino or piperidino radical; S or a substituted sulfurous group; a halide, preferably an F, Cl, or CF₃ group; substituted or unsubstituted alkyl groups, wherein the substitutions can be any element or group, including, but not limited to, those that form a heterocyclic radical, such as a morpholino or piperidino radical; O-alkoxy, wherein the alkoxy group is a C₁-C₁₈, preferably a C₁-C₁₂, and more preferably a C₁-C₆ alkoxy, such as methoxy or ethoxy; oxime or formamide groups; substituted or unsubstituted aryl group, such as a phenyl or naphthyl group, wherein the substitutions can be any element or group, including, but not limited to, alkyl or alkoxy groups (e.g., methyl, ethyl, propyl, isopropyl, butyl, isobutyl, sec-butyl, tert-butyl, methoxy, ethoxy, propoxy, isopropoxy, butoxy, isobutoxy, sec-butoxy, or tert-butoxy) and those that form a heterocyclic radical, such as a morpholino or piperidino radical; substituted and unsubstituted alkenyl; substituted and unsubstituted cyclic alkyl, substituted and unsubstituted halide; acyl, alkylsulfoxyl, alkylsulfonyl, alkylthiol, hydroxyl, thiols; symmetrical and optical isomers thereof; mixtures thereof; and salts thereof.

Other non-stilbene compounds are also provided by the invention. These compounds are identified through their action on the activity of one or more AT, and preferably also on their effect on the activity and/or viability of one or more nematode, insect, or Acari. Non-limiting exemplary compounds are those disclosed in the priority document U.S. provisional patent applications.

Thus, compounds can have structures other than those generally recognized as stilbenes. Such compounds are identified by the methods of the invention and can provide the same benefits and nematicidal, insecticidal, and acaricidal activities as the stilbene compounds disclosed herein.

A basis of the invention derives from studies showing that a stilbene natural product, DST, (FIG. 1B) could be isolated from the symbiotic bacterium Photorhabdus luminescens, which lives inside nematodes of the genus Heterorhabditis (Hu et al., 1999). DST was found to be a major bacterial metabolite, present at about 3 mg/g of nematode-infected Galleria mellonella larvae (Hu et al., 2000). This compound has nematicidal activity against a variety of nematode species (24 hr lethality 100% at 100 ug/ml), including Caenorhabditis elegans and Meloidogyne incognita (Hu et al., 1999). Interestingly, the symbiotic nematodes within which the bacterium lives (Heterorhabditis spp., in this case H. megidis), were completely insensitive to DST at concentrations up to 200 ug/ml (Hu et al., 1999). The authors presenting those findings made no claims as to the mode of action of DST. But later it was disclosed that the therapeutic action of this stilbene compound relates to its effectiveness as a protein kinase inhibitor, and it was proposed that DST would be a useful drug for the treatment of human inflammatory diseases (Chen et al., 2001).

Another basis of the invention derives from the fact that the particular stilbene compound DIDS (FIG. 1C) was reported to possibly be a blocker of anion transporters (Cabantchik and Greger, 1992). However, although the specific DIDS compound was proposed to act on anion transporters, toxicity resulting from this molecular effect was not reported. That is, although this particular stilbene compound was known to have inhibitory effects on anion transporters, it was not known to be useful as a killing agent or deterrent agent against living organisms (e.g., pests). Furthermore, no particular activity was assigned to the general stilbene structure.

In developing the present invention, the inventors have recognized that DST bears strong structural resemblance to DIDS (see FIGS. 1B and 1C). In view of this structural similarity, the inventors hypothesized that the two compounds would share a similar mode of action on anion transporters on cellular membranes, and that this molecular mode of action could be the basis of physiological insecticidal or nematicidal activity. The present invention is thus predicated, at least in part, on the discovery of the nematicidal activity of stilbene compounds, acting through disruption of anion transporter activity. Based on this realization, the inventors recognized the utility of AT for screening for other compounds having AT-disrupting (activating or inhibiting) activity, with the realization that such compounds would have nematicidal, insecticidal, and/or acaricidal activity. Further, the inventors have recognized that the use of a particular AT from a particular nematode, insect, or Acari can be used to identify lead compounds, pesticides, etc. that are specific or semi-specific for the organism from which the AT was derived, and thus provide a lead compound, pesticide, etc. that can be used to specifically treat infestation or parasitism by a particular organism while having little or no effect on other organisms. For example, identification of a compound that is active against a particular AT from a plant parasitic nematode can permit production and use of a pesticide that is active against that parasite while leaving free-living, potentially beneficial nematodes unharmed. In embodiments, the nematode is not a pine wood nematode.

In a second aspect, the present invention provides compositions comprising compounds having anti-nematicidal activity, anti-insecticidal activity, anti-Acari activity, or activity against two or all three types of these organisms. The compositions can be used for treatment of plants, animals, or the environment surrounding selected plants and/or animals. In general, the compositions comprise one or more compounds having nematicidal activity, insecticidal activity, acaricidal activity, or activity against at least one particular species of two or all three types of these organisms, along with another substance. In embodiments, the compound has nematicidal activity against one or more nematodes. In embodiments, the compound has insecticidal activity against one or more insects. In embodiments, the compound has activity against one or more nematode and one or more insect. Thus, the present invention provides for use of one or more compounds, such as stilbene compounds, as insecticides, nematicides, and acaricides, and to prepare insecticides, nematicides, and acaricides.

In the compositions, the compound is present in an amount or concentration that is sufficient such that, when applied to a plant, animal, or the environment (including an artificial, in vitro, or laboratory or research environment) shows a measurable effect on the growth, survival, or reproduction of at least one nematode, insect, or Acari. Preferably, the compound is present in a sufficient amount to kill at least some of the nematodes and/or insects and/or Acari within the area treated with the composition. In preferred embodiments, the compound is present in a sufficient amount or concentration to kill a majority, essentially all, or all of the target organisms in the area treated. As should be evident, the compound(s) may be selective for nematodes, insects, or Acari, or specific for one or a few species within one, two, or all three of these groups. Thus, they may be selective for one or more particular species of nematodes and/or one or more specific species of insects. These species are referred to herein as “target” species. Accordingly, the amount of compound included in the compositions can be sufficient to kill some, most, or all of one species of nematode (or insect or Acari), yet be less effective against some or all other species of nematodes (or insects or Acari). In general, the active compound is present in the composition in an amount that is sufficient such that, when applied to a plant, animal, environment, nematode, insect, or Acari, it is present in a concentration of 500 parts per million (ppm) or less. In embodiments, the concentration when applied is 100 ppm or less, such as 50 ppm, 25 ppm, 10 ppm, 5 ppm, 2 ppm, 1 ppm, or even less. Thus, in embodiments, it is 500 parts per billion (ppb), 250 ppb, 100 ppb, 50 ppb, 10 ppm, or less. In general, because each particular compound will have different specific activities, and each might be used to treat multiple target organisms, each with different sensitivities to the compound, a broad range of useful concentrations is envisioned by the invention. For example, a working concentration of from 10 ppb to 10 ppm is suitable. Likewise, a range of from 10 ppb to 1 ppm is suitable, as is a range of from 100 ppb to 10 ppm, or a range of from 10 ppm to 100 ppm. Compositions having much higher concentrations of the compound(s) of the invention are contemplated, with reduction in concentration prior to use, such as by dilution, being recommended. Thus, for example, a concentrated composition of 6 pounds of compound per gallon of solution, 4 pounds of compound per gallon of solution, or 1 pound of compound per gallon of solution can be provided.

The compositions comprise the compound and at least one other substance. The other substance can be any suitable substance. Thus, it can be water or another solvent that is useful for creating a liquid composition. It can be a compatible carrier or binder, such as is commonly employed in products for use in agriculture and animal husbandry settings. As the compounds can be used to treat nematodes and insects affecting humans, the other substance may be a pharmaceutically acceptable substance, such as those that are well known in the medical arts. Other non-limiting substances of particular note are dispersants or other substances that aid in distribution of compositions in agriculture settings. These can be biologically active or inactive substances that act as fillers or dilution agents. They can also be compounds that improve the solubility of the compounds in aqueous environments. In preferred embodiments, the compositions comprise one or more substances that improve the solubility of the compound in water or an aqueous liquid.

Of course, the composition may comprise the compound and only one other substance. Alternatively, it can comprise the compound and two or more other substances. In embodiments, one of the other substances is another compound having nematicidal and/or insecticidal activity, including a substance(s) identified using the methods of the present invention. In yet other embodiments, the composition comprises one or more other substances that have biological activity, but not nematicidal or insecticidal activity. For example, the composition may comprise one or more nematicidal and/or insecticidal compounds and one or more substance that is beneficial for plant or animal growth, such as a fertilizer (e.g., a nitrogen, phosphorous, potassium, calcium, magnesium), or one or more substance that is harmful for plant or animal growth, such as a herbicide (e.g., glyphosphate).

Compositions according to embodiments of the present invention comprise one or more stilbene compounds. In embodiments, they do not comprise DIDS or DST. They also may comprise substances that are compatible with the stilbene compounds. Such substances can be those suitable for stabilization of the stilbene compounds or suitable for increasing the solubility of one or more stilbene compounds in water or an aqueous composition, such as a solution, dispersion, and the like. They can also be substances that are biocompatible and suitable for administering (either internally or topically) to a multicellular organism that serves as a host organism for a nematode or insect, such as a plant, fish, farm animal, companion animal, or human. Such compounds are well-known to those of skill in the agricultural, aquaculture, veterinarian, and medical professions, and need not be detailed here. Examples include, but are not limited to, binders, fillers, colorants, and surface active agents. Examples also include, but are not limited to, bioactive agents, such as antibiotics, antifungals, hormones, nutrients, vitamins, and the like. Further examples include, but are not limited to, synergists that block metabolism, such as monooxygenase inhibitors (e.g., piperonyl butoxide). Compositions of the invention may also include a solvent, such as water or a water-organic solvent.

In a third aspect, the present invention provides a method of treating at least one plant, at least one animal (including humans), and/or at least one environment. The method generally comprises exposing the plant, animal, and/or environment to at least one compound in a sufficient amount to inhibit the activity or viability of at least one nematode or insect or Acari. In embodiments, a sufficient amount is provided to directly kill at least one nematode or insect. In preferred embodiments, the method generally comprises exposing at least one plant, at least one animal, and/or at least one environment to at least one compound in a sufficient amount to inhibit the activity or viability of at least one nematode and/or insect, or a sufficient amount to directly kill at least one nematode and/or insect, where the compound is identified through a method of identification or screening according to the present invention. Thus, the invention provides for use of compounds and compositions of the invention for production of one or more insecticides and nematicides. In embodiments, the method kills or inhibits the activity of a free-living nematode. In embodiments, the method kills or inhibits the activity of a plant parasite.

In embodiments, the method treats at least one plant. In embodiments, the method treats at least one animal. In embodiments, the method treats at least one human. In embodiments, the method treats the environment surrounding at least one plant and/or at least one animal. Where the subject treated is a plant, the plant can be any plant. It thus can be a plant having agricultural value or being raised in an agricultural setting. Examples include, but are not limited to, plants for human consumption (e.g., corn, oats, wheat, barley, hops, and other grains and/or grasses), green vegetables (e.g., beans or other legumes, lettuces, cabbages, asparagus), tomatoes, onions, carrots, melons, gourds, and berries (e.g., blueberries, strawberries, raspberries, blackberries). The plant may also be a floral plant, such as a rose, tulip, lily, hydrangea, violet, orchid, and the like. Furthermore, the plant may be a landscaping plant, such as those typically sold by nurseries and home centers (e.g., maple, oak, pine, shrub, and the like). Where the subject treated is an animal, it can be any animal. In some embodiments, it is a mammal. In embodiments, it is specifically a human. It thus can be an animal that is grown or used in an agriculture setting, such as on a farm or ranch. It thus can be a horse, cow (e.g., cattle, dairy cow), sheep, goat, or pig. It can also be a fish grown in aquaculture, such as a trout, or catfish. It likewise can be a bird, such as one used for animal or human food, including, but not limited to, chicken, turkey, duck, and goose. An animal that is treated may also be a companion animal, such as a dog, cat, or bird.

When the method is practiced to treat at least one environment, it can be any environment. Thus, the environment can be a natural environment or one that is man-made. Non-limiting examples of environments that can be treating are agriculture environments (e.g., a farm or ranch), a research plot containing one or more types of plants or animals upon which research is being performed, a freshwater stream, river, pond, or lake, whether it be above or below ground. In view of the above utilities, it should be evident that the present invention provides for the use of compounds, such as stilbene compounds, for the treatment of one or more plants, animals, or environments to reduce or eliminate at least one nematode and/or at least one insect and/or at least one Acari.

Typically, in the method, the plant and/or animal treated is one that is parasitized, infected, damaged, or killed by one or more Acari, insect, and/or nematode species, including, but not limited to nematodes. The method also may be practiced on at least one plant, animal, and/or environment that, while not currently being parasitized, infected, damaged, or killed by a nematode and/or insect and/or Acari, is known to be susceptible or often parasitized, infected, damaged, or killed by one or more of these organisms.

The method also comprises treating plants and animals that are bitten, damaged, or killed by one or more Acari and/or insect and/or nematode species. Inhibition can be by reducing the viability of the organisms, resulting in ultimate death of the organisms, or by direct killing of the organisms. Alternatively, it may simply reduce the activity of the organism, and thus reduce damage caused by that organism. A reduction in activity of nematodes and/or insects and/or Acari, while not completely eliminating a source of economic loss, still provides an economic benefit by reducing damage caused by these organisms.

As a general matter, the method comprises the step of exposing. The step of exposing can be any activity that results in contact of the compound with the plant, animal, and/or environment. The compound can thus be contacted with the plant, animal, or environment directly or indirectly. Contact can be, for example, by spraying, dusting, dipping, fogging, misting, watering, fumigating, injecting, ingesting, and rubbing. It thus can be by crop dusting. It also can be by broadcasting on an agricultural environment prior to planting of a crop or allowing animals to graze. One non-limiting example of exposing is adding a compound, such as a stilbene compound, to an environment, and permitting natural dispersion of the compound to effect contact (e.g., add a stilbene to an aqueous environment containing a nematode, and permit diffusion of the stilbene to effect contact with the nematode). Where the method is practiced in vitro for research purposes, the method can comprise providing an environment containing the compound, and placing the nematode or insect or Acari in that environment. For example, it can comprise providing a culture dish having the compound of interest attached, adhered, or otherwise associated with the surface of the dish or a medium in the dish, then introducing the nematode or insect into the dish.

Inhibiting activity is a broad term that generally denotes affecting the normal life and life processes of a nematode or an insect or an Acari. It thus can affect the metabolism of an nematode or insect. It likewise can affect the growth and/or development of the organism. It can affect the sexual development of the organism. In embodiments, inhibiting can be considered the act of reducing growth of one or more nematodes or insects or Acari from an immature to a mature stage, reducing the ability to reproduce or the rate of reproduction (as compared to untreated nematodes or insects of the same species in the same environment), reducing the amount of feeding or the ability to metabolize food. In certain embodiments, inhibiting is the act of causing the death of at least one nematode, at least one insect, at least one Acari or at least one of each. The act of inhibiting can cause a result in a short period of time or over a prolonged period of time. Thus, the action of the compound(s) can be rapid (less than one day), or prolonged (more than two weeks). Preferably, sufficient effect on activity is seen in one week or less, such as 7 days, 6 days, 5 days, 4 days, 3 days, 2 days, or 1 day. About 72 hours, 60 hours, 48 hours, 36 hours, 30 hours, 24 hours, 20 hours, 16 hours, 12 hours, 8 hours, 6 hours, 4 hours, 3 hours, 2 hours, 1 hour, or less. 60 minutes or less, 30 minutes, 10 minutes, 5 minutes or less. 60 seconds or less—30 seconds or less, 15 seconds or less.

According to the invention, a direct effect is by direct contact of the compound with the nematode and/or insect and/or Acari, while an indirect effect is by contact of the compound with a plant, animal, or environment, then contact of a nematode or insect or Acari with that plant, animal, or environment. Thus, direct killing is by contact of the compound with the organism directly from the source of exposure (e.g., direct contact upon spraying from an airplane, a truck-based container, a hand-held sprayer, or a can, such as a hand pump). Likewise, indirect killing is by application to environment or animal or plant, then contact with a target organism by contact of the target organism with the treated plant or animal or environment. Accordingly, indirect killing can occur at a time that is considerably different than the time at which the compound was exposed to the plant, animal, or environment. Indirect treating is exemplified in one embodiment by treatment of a plant, then eating of plant material by a target organism.

The compound may be exposed to the plant, animal, and/or environment as the sole substance provided. It may also be exposed in conjunction with another compound according to the invention. The nematicidal and/or insecticidal and/or acaricidal compound(s) may be provided and used as purified (partially, essentially completely, or completely) products, or may be provided as part of one or more compositions. Where one or more compositions are provided, they may be any of those discussed above. Of course, one or more compounds may be provided, each as purified products, all together in a single composition, or some as purified products and others in a composition. As should be evident, the method of treating can comprise performing the exposing step more than one time. Thus, the method contemplates a regimen where the plant, animal, or environment is treated multiple times. The time interval between exposures will depend on the plant, animal, and environment, as well as the level of infestation of nematodes and/or insects and/or Acari, and the amount or concentration of compound used. In general, it is preferred that, for agriculture purposes, a single exposure is performed per growing season. Two or more exposures are also contemplated. Alternatively, exposure of a plant, animal, or environment can be performed on a recurring basis, such as once a year, once every four months, once every three months, once every two months, or once a month. Exposure can also be repeated as needed, based on visual observation (e.g., when damage of crops is observed). Practitioners may select the most appropriate exposing regimen for each particular application of the method of the invention.

In embodiments, the present invention provides methods of treating multicellular host organisms that are currently parasitized or susceptible to being parasitized by a nematode or insect or Acari. The methods of treatment of these organisms generally comprise contacting the host organism with at least one compound, such as a stilbene compound, in amount sufficient to disrupt the activity of at least one anion transporter of the nematode or insect or Acari, the disruption blocking or causing a reduction or cessation of parasitism of the target organism on the multicellular host organism. The compound can be applied directly (either topically or internally) to the host organism or to the environment surrounding the host organism. The effect on a target organism of contacting the compound to the host organism occurs upon contact of the target organism with the host organisms, and entering of the compound into the target organism. In embodiments, the method blocks an attack on the host organism by a target organism by interfering with AT function before the target organism can parasitize the host. In embodiments, the method reduces or eliminates an attack on the host organism after it has begun by interfering with AT function of the target organism that is attacking or parasitizing the host. In embodiments, the method ends an attack or prevents further attack by killing the target organism by disrupting function of at least one AT. In embodiments, the methods do not include treating with DST. In embodiments, the methods do not include treating with DIDS. In embodiments where treatment is directed at insect damage, the method does not include treating with a compound that affects a ligand-gated chloride channel of the insect. Thus, the invention provides for use of compounds, such as stilbene compounds, for treatment of plants, animals, and/or environments to reduce or eliminate at least one nematode and/or at least one insect or Acari. It accordingly provides for use of compounds, such as stilbene compounds, for treatment of at least one nematode and/or at least one insect and/or at least one Acari, to reduce its viability or kill it.

The present invention also provides methods of protecting a multicellular host organism parasitized by a nematode or insect or Acari, or susceptible of being parasitized by a nematode or insect or Acari. These methods comprise contacting the target organism with at least one compound, such as a stilbene compound, in amount sufficient to disrupt the activity of at least one anion transporter of the target organism. Contacting the target organism, such as a nematode, with the compound disrupts the function of at least one AT, causing a reduction or cessation of parasitism of the target organism on the multicellular host organism. In embodiments, the method does not include treating with DST. In embodiments, the method does not include treating with DIDS. In embodiments where the method is a method of protecting a host from an insect, the method does not comprise treating with a compound that acts on a ligand-gated chloride channel of the insect.

In view of the above methods, it is evident that the present invention provides a method of treating where both the host organism (or its environment) and the target organism are treated simultaneously with the same act of contacting. It likewise provides a method of killing target organisms by contacting them with at least one compound, such as a stilbene compound. It further provides a method of altering (i.e., inhibiting or activating) the activity of at least one AT by exposing the AT to, or contacting the AT with, at least one compound, such as a stilbene compound.

In exemplary embodiments, the compound is a stilbene compound. In preferred embodiments, the stilbene compounds used for contacting a host organism, a nematode or insect or Acari, or an AT (and the compositions comprising them) do not comprise DST. In embodiments, the compounds do not comprise DIDS.

In methods of treating plants, compounds, such as stilbene compounds, are typically applied directly to the target plant or to the soil surrounding the target plant, and the target organism is exposed to the compound as a result of contact with the treated plant or soil. Other methods include treating water in which the target organism lives (during at least a portion of its life cycle), and treating the food of a fish or animal, or treating the fish or animal itself. In certain methods, both the plant, fish, or animal and the target organism are exposed at the same time. The methods of the invention rely, at least in part, on the activity of the compounds of the invention on one or more AT of the target organism, where such activity disrupts normal function of the AT, resulting in impaired anion transport, loss of cellular function, and reduction in viability, or death, of the target organism. The methods of treating may be methods of reducing or blocking initial parasitic activity of nematodes or insects (i.e., protective or prophylactic methods of treating plants, fish, animals), methods of reducing or ending active attacks, or methods of eliminating nematodes or insects from plants, fish, or animals.

In another aspect, the invention provides methods of screening for compounds, such as particular stilbene compounds, that show nematicidal and/or insecticidal and/or acaricidal activity. The methods can comprise screening for compounds that affect the activity of at least one AT, and have nematicidal and/or insecticidal activity. The methods use one or more AT as screening agents to detect compounds that bind to and affect the activity of the AT. The methods generally comprise contacting one or more AT molecules with one or more compounds and determining if the activity of the AT was altered, alteration of activity indicating that one or more of the compounds affected the activity of the AT. The methods of screening are based, at least in part, on the discovery of the effect of inhibition of AT by stilbene compounds on parasitic activity and viability of nematodes and insects. Accordingly, the invention provides for use of AT to identify compounds having nematicidal and/or insecticidal and/or acaricidal activity, or activity that reduces the viability of one or more nematodes and/or insects and/or acarines.

In the method of screening, the step of contacting can comprise any activity that results in physical contact of at least one test compound with at least one AT molecule. As used herein, a test compound is a compound of known or unknown structure that is being investigated for its potential affect on one or more AT. As the AT and test compound are relatively small compared to the devices available for physical manipulation of substances, the act of contacting typically comprises adding the compound to a composition comprising the AT. For example, an AT can be present in a reaction mixture, and the test substance is added to the mixture. Sufficient time is provided for the test substance and the AT to come into contact, then the activity of the AT is assayed.

Numerous AT assay compositions are known in the art, and any of them may be used in accordance with the invention. Depending on the particular assay selected, the amount of time might vary; however, those of skill in the art are well aware of suitable times for contact to occur within the context of each of the various possible protocols known in the art. Exemplary reaction assays and conditions are provided in the Examples, but other, equally effective conditions and methods are known to those of skill in the art from published journal articles. For example, AT activity can be assayed using electrophysiological methods to measure blockage of ion currents through AT according to Machaca, K., et al., “A novel chloride channel localizes to Caenorhabditis elegans spermatids and chloride channel blockers induce spermatid differentiation”, Dev. Biol. 176(1):1-16, 1996. Alternatively, one may use ³⁶Cl-ion flux assays to determine chloride ion movements through AT by biochemical studies according to Payne, G. T. and Soderlund, D. M, “Activation of gamma-aminobutyric acid insensitive chloride channels in mouse brain synaptic vesicles by avermectin B1a”, J. Biochem. Toxicol. 6:283-292, 1991. Other assays can include use of fluorescence methods to measure, directly or indirectly, effects on AT function. Effects on AT function can directly alter the fluorescence signal of dyes like MQAE (see, for example, Munkonge F, et al., “Measurement of halide efflux from cultured and primary airway epithelial cells using fluorescence indicators”, J. Cyst. Fibros. 2004; Suppl 2:171-6). In addition, AT blockage can also upset the acid balance of a cell, leading to a change in intracellular pH, and this effect could be used in a pH-dependent fluorescence-based assay according to, for example, Vieira L. et al., “Chloride conductive pathways which support electrogenic H+ pumping by Leishmania major promastigotes”, J Biol Chem. 1995; 270 (10): 5299-304. Furthermore, one can screen candidate molecules for their ability to displace radioligands, such as [³⁵S]TBPS from the AT according to the method of, for example, Abalis, I., et al., “Binding of GABA receptor channel drugs to a putative voltage-dependent chloride channel in Torpedo electric organ”, Biochem. Pharmacol. 34:2579-2582, 1985.

Many of the published protocols are specific for in vitro assays; however, it is to be understood that the present invention comprises both in vitro and in vivo screening assays, including those that comprise both in vitro and in vivo aspects. Although the methods can be practiced in vivo, typically they are performed in vitro, or initially performed in vitro, with confirmatory assays performed in vivo. Where both in vitro and in vivo assays are performed, it is preferred that the in vitro assays precede the in vivo ones.

The method of screening according to the invention can comprise contacting one or more AT with one or more test compounds in vitro, followed by contact of one or more AT with one or more test compounds in vivo. Thus, the methods of the invention can comprise contacting at least one test compound and at least one living nematode or insect. When performed in vivo, the effects of the contacting can be determined by observing the activity or viability of the nematode or insect or acarine. It thus can include determining whether a nematode or insect is killed by the contact.

In preferred embodiments, the method comprises contacting at least one AT from a known species of nematode, insect, or Acari with at least one compound, and determining the effect of the compound(s) on the AT(s). Where more than one compound is screened at a time, and at least one is determined to have an effect, the compounds are re-screened until each active compound is identified. Likewise, if more than one AT is used in the initial screening, the number of AT used in subsequent screenings is reduced until a the loss in activity can be assigned to one or more particular AT. Ultimately, the effect of each active compound (also referred to herein as a lead compound) individually on each AT individually can be tested to find specific relationships between particular compounds and particular AT.

In embodiments, a particular active compound is found to have an effect on a particular AT. In such embodiments, the method can further comprising correlating the activity on the AT with the inhibitory or killing effect of the compound on a target organism in vivo. Due to the recognition of the molecular basis of the effects of AT-inhibiting compounds on target organism activity and viability, this embodiment of the method provides a confirmatory correlation for the in vivo activity of the active compound. Correlation not only confirms the activity of the lead compound in vivo, but permits one to determine the specificity of the lead compound for various species of target organisms. In vivo inhibition/killing assays are known in the art, and exemplary assays are provided herein. Any suitable in vivo inhibition/killing assay may be used in this embodiment of the method of the invention, the choice of any particular series of steps being well within the competency of those of skill in the art, and being made based on any number of parameters, including, but not limited to, cost, time, availability of reagents and supplies, etc.

The method of screening comprises using one or more AT molecules. In embodiments, it comprises using a single AT molecule (typically, a combination of numerous AT molecules, which are all of the same type/amino acid sequence). In other embodiments, it comprises using two or more AT molecules. As used herein, when substances (whether it be a test compound, an AT, or any other substance) are referred to as being present or being present as “a single” substance, it is meant that a substance having a particular identity is present, in one or multiple identical or essentially identical copies. Thus, stating that the method comprises using a single AT means that an AT with a particular amino acid sequence is used, and that the AT is present in a single or multiple (up to millions or billions) of copies. According to the method of screening, test compounds can be contacted with multiple different AT molecules, then re-screened with a subset of those AT molecules to identify which AT molecules are being affected. Ultimately, the method can comprise screening with a single AT molecule. Where more than one AT is used in the method, the method may further comprise exposing the compound to each AT individually to determine which AT is affected.

The method of screening comprises using one or more test compounds. In embodiments, it comprises using a single test compound. In other embodiments, it comprises using two or more test compounds. The method can comprise determining a test compound after a single iteration of the screening process. Alternatively, according to the method of screening, multiple different test compounds can be contacted with one or multiple different AT molecules, then one or more subsets of the test compounds showing activity on the AT can be re-screened with the same AT, a subset of the AT, one or more different AT, or a mixture of some or all of the same AT and one or more different AT (preferably the same set of AT used in the first screen). One or more sub-subsets can then be contacted with an AT or mixture of AT (preferably from the same set of AT used in the first and/or second screen) to identify which AT molecules are being affected. Re-screening can be repeated until a suitable number of test compounds are determined. Ultimately, the method can comprise screening with a single AT molecule and/or screening a single test compound. Where more than one AT is used in the method, the method may further comprise exposing the compound to each AT individually to determine which AT is affected. For example, where more than one stilbene test compound is used, the method may further comprise exposing the AT to each stilbene individually to identify a stilbene having the desired activity. In embodiments, the method of screening is a high-throughput method. Those of skill in the art are well aware of the parameters for screening numerous compounds in a high-throughput assay format; therefore, the details of such assays need not be detailed here.

The method of screening can be practiced on compounds having any structure because the method relies on the activity of one or more AT for identification of active compounds. However, in embodiments, the likelihood of finding a suitable compound is increased by screening compounds having the general structure of a stilbene (FIG. 1A). While this structure is not required for activity, it is likely that compounds having this core structure will be more likely to have the desired activity than compounds having randomly selected structures. With the power of high-throughput screening and combinatorial chemistry, this advantage is not as evident. However, in embodiments where less powerful techniques of screening are used, focusing on the stilbene core structure can provide a cost and time benefit.

In embodiments, the stilbene compound is not DIDS, and the compounds being screened do not include DIDS. In embodiments, the stilbene compound is not DST, and the compounds being screened do not include DST. In embodiments, the stilbene compound is not a disulfonic acid stilbene, and the compounds being screened do not include a disulfonic acid stilbene. In embodiments, the AT is not a ligand-gated chloride channel of an insect.

The method of screening comprises determining if the activity of the AT was altered. An AT has an altered activity if its activity is detectably different in the presence of one or more test compound than in the absence of the compound. The difference can be determined using any of a number of assays, as known in the art and disclosed herein. Comparison can be made between the same AT (e.g., determine activity, then add test compound(s) and determine activity again) or between an AT in one reaction vessel containing the test compound and an identical AT, in the same composition as the first AT but without the test compound.

While it is preferred that contacting result in the activity of the AT being inhibited, diminished, etc., the methods of the invention may also identify compounds that activate, enhance, etc. the activity of one or more AT. Such activators may be used for various purposes, including, but not limited to, use as competitors for characterization of certain AT inhibitors, and use as nematicides and as insecticides. The disruption of AT activity, whether inhibition or activation, disrupts normal cellular activity and result in loss in the target nematode and/or insect activity, loss in viability, and/or death.

The method of screening, in its basic form, comprises contacting at least one AT with at least one test compound, and determining if the compound affects the activity of the AT. In embodiments, the method further comprises identifying the test compound(s) that affect the AT. Where more than one AT is used, the method can further comprise identifying which AT is affected by which test compound (if more than one test compound is used). Identifying the test compound showing activity can be through any of the various methods used by those of skill in the art. Of course, if the identity of the test compound was known prior to performing the method of screening, it is a simple matter to identify the test compound showing activity. Where the identity was not known prior to practicing the method, it can be determined by mass spectroscopic analysis, chemical degradation, chromatographic techniques, IR spectroscopy, NMR, and the like. Where multiple test compounds were contacted with the AT, the method can comprise identifying one, some, or all of the test compounds showing activity. In such embodiments the method can comprise separating each test compound from each other.

The method can further comprise testing one or more test compounds showing in vitro activity for in vivo activity. For example, it can comprise contacting a positive test compound with one or more nematodes in a culture dish. Alternatively, it can comprise contacting a positive test compound with one or more nematodes in a natural environment, such as an agriculture plot. The effect of the positive test compound on the nematode (or insect, if practiced in vivo on an insect) can be determined. Determining can be by visual observation of the activity of the nematode (or insect), or by assaying any of a number of cellular processes indicative of the health and viability of the nematode (or insect). If desired, the method can comprise a large test, such as a field test on an agricultural plot.

Ultimately, the method of screening can provide the practitioner with a compound of known structure and activity. The invention provides for modification of the positive test compound to engineer a compound having one or more altered activity. For example, the method can comprise engineering a positive test compound to have a higher specificity for a particular AT, to have broader specificity, to have lower toxicity in aqueous environments, etc. Screening of the modified compound can be accomplished using the methods of screening of the invention. Because the method of the invention can identify active compounds, which can then be modified and re-screened, the method of the invention can be a method of identifying lead compounds for use as nematicides or insecticides or acaricides.

The method can include one or more control reactions to determine if one or more of the steps of the assays were performed properly and/or to determine if one or more of the reagents functioned as expected. Those of skill in the art are well aware of the parameters for conducting such control reactions, and thus the details need not be disclosed here. In one embodiment, free-living or otherwise non-harmful species of nematodes, insects, or Acari are used as a control to determine if the test compound(s) affect the viability of the free-living or non-harmful organism. This information can be beneficial in selecting lead compounds for continued research.

### EXAMPLES

The invention will be further explained by the following Examples, which are intended to be purely exemplary of the invention, and should not be considered as limiting the invention in any way.

### Example 1

Identification of Nematicidal Activity of DIDS

Using the known susceptibility profile of certain nematode species for DST as a guide for selecting species to test, DIDS was tested for toxicity in nematodes. Based on the profile for DST, it was hypothesized that M. incognita would be susceptible to inhibition by DIDS, and that H. bacteriophora would be resistant (H. bacteriophora is a close relative of H. megidis that also carries Photorhabdus luminescens). It was postulated that, if AT are involved in toxicity, DIDS should be toxic to the plant parasitic nematode M. incognita and inactive to the symbiont-containing nematode H. bacteriophora.

The methods for culturing the nematodes and the toxicity assays we performed as follows:

Experimental Methods for the Toxicity Studies:

Culture Methods

Heterorhabditis bacteriophora was cultured using Galleria inellonella L. (Lepidoptera: Pyralidae) as a host insect. Briefly, insects were infected by exposing them to about 100 infective stage juvenile nematodes per insect in a Petri dish lined with filter paper. After about 2 days, the insect dies and is transferred to an emergence trap, called a White trap. This trap is a 60 mm Petri dish lined with filter paper to hold the cadaver, which is floated on water in a 90 mm Petri dish. After approximately 2 weeks, the infective juveniles exit the cadaver and crawl from the small Petri dish to become entrapped in the water held in the larger dish.

Meloidogyne incognita cultures were maintained on potted tomatoes (c.v. Rutgers) in a greenhouse. Infective juvenile nematodes for analysis were extracted as eggs from infected tomato roots by shaking the roots in 1% bleach for 2 minutes. Eggs were captured in a 500-mesh sieve, rinsed in water and placed in Baerman funnels. When the eggs hatched, the juveniles moved downward in the funnels, and hatched infective juveniles were collected after 48 hours.

In vivo Assays of Toxicity

We determined the toxicity of DIDS to infective juveniles of M. incognita and H. bacteriophora. Infective juveniles were assayed because they represent the only free-living life stage for both nematode species; adults live within their respective hosts and are not amenable to this type of test. Further, the infective juvenile of M. incognita would likely be the target of a nematicide based upon this material. The tests were conducted in either 35 mm Petri dishes or 96 well plates, holding the test material dissolved in DMSO and dispersed into tap water. Each dish was considered a replicate and contained 10-30 nematodes of either species. Each well of the 96-well plates had one nematode.

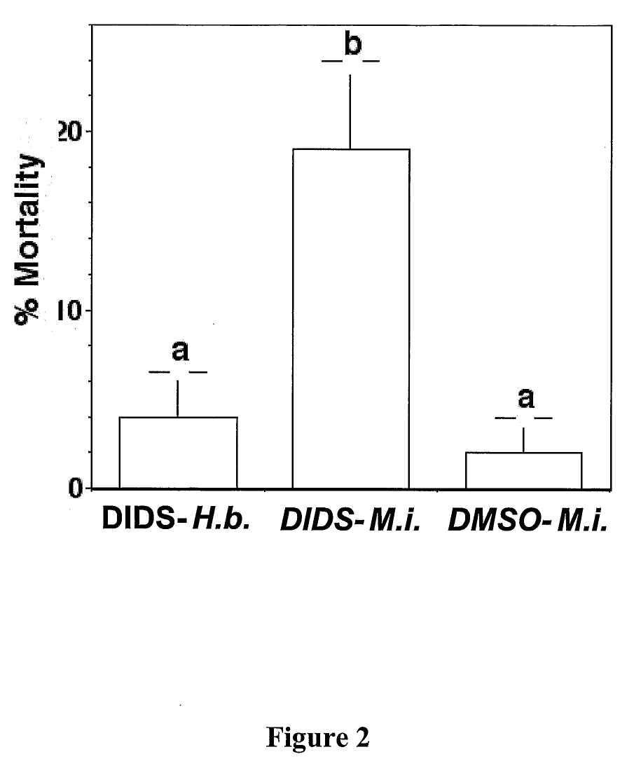

FIG. 2 shows the results of a 24 hr toxicity bioassay of DIDS (100 ug/ml, 200 uM) to nematodes. DIDS-H.b.=DIDS-exposed H. bacteriophora; DIDS-M.i.=DIDS-exposed Meloidogyne incognita. DIDS was dissolved in DMSO, and DMSO-M.i.=DMSO-exposed Meloidogyne incognita. Average percentage mortality for each treatment group was calculated from 5 replicates containing 10 nematodes each. Bars are means±SEM. Letters indicate results of ANOVA (p<0.008) and Student-Newmann-Keuls means separation test, where bars labeled by a different letter are significantly different (p<0.05).

DIDS was dissolved in DMSO, and this vehicle alone had little or no effect on nematode survival. In contrast, DIDS proved to be paralytic/lethal to M. incognita, a plant pest nematode, but not to H. bacteriophora. Thus, DIDS shows a cross resistance pattern similar to that previously observed for DST. These results demonstrate, for the first time, a linkage between nematode lethality and chemistry known to affect AT channel function. These findings are especially compelling because they demonstrate a bona fide linkage between mode of action and in vivo toxicity.

### Example 2

Effect of DIDS on a Plant Parasite and on a Free-Living Nematode

To further characterize the nematode-inhibiting effect of a stilbene compound (DIDS), experiments were performed as described above, using a 96 well plate and 10-30 nematodes per well, and using the plant parasite M. incognita and the free-living C. elegans. The results are depicted in Tables 1 and 2.

TABLE 1
Toxicity of DIDS On C. elegans

| Day | LC₅₀ (ppm) | 95% CI | Slope | χ² (df) |
|---|---|---|---|---|
| 1-day (24 hr) | 196.13 | 89.67-1632.87 | 1.43 ± 0.35 | 0.24 (3) |
| 2-day (48 hr) | 83.70 | 52.20-226.34 | 1.45 ± 0.29 | 0.46 (3) |
| 3-day (72 hr) | 31.53 | 25.12-42.87 | 1.87 ± 0.28 | 0.41 (3) |
| 5-day (120 hr) | 15.69 | 13.05-18.37 | 2.86 ± 0.36 | 0.32 (3) |
| 7-day (168 hr) | 10.67 | 8.81-12.22 | 4.57 ± 0.71 | 2.44 (3) |

TABLE 2
Toxicity of DIDS On M. incognita

| Day | LC₅₀ (ppm) | 95% CI | Slope | χ² (df) |
|---|---|---|---|---|
| 1-day (24 hr) | 160.03 | 78.92-1668.69 | 1.77 ± 0.50 | 2.47 (3) |
| 2-day (48 hr) | 97.91 | 59.37-277.45 | 1.52 ± 0.30 | 2.27 (3) |
| 3-day (72 hr) | 37.84 | 29.51-54.98 | 1.83 ± 0.30 | 0.44 (3) |
| 5-day (120 hr) | 17.07 | 14.23-20.00 | 2.75 ± 0.36 | 0.33 (3) |
| 7-day (168 hr) | 11.58 | 6.72-15.02 | 4.04 ± 0.59 | 4.16 (3) |

As can be seen from the tables, DIDS was found to be toxic to both free-living nematodes and plant parasite nematodes, producing paralysis in 2 days. It has been found that DIDS at 10 ppm is toxic to these organisms in as little as 2 days.

It will be apparent to those skilled in the art that various modifications and variations can be made in the practice of the present invention without departing from the scope or spirit of the invention. Other embodiments of the invention will be apparent to those skilled in the art from consideration of the specification and practice of the invention. It is intended that the specification and examples be considered as exemplary only.

### REFERENCES CITED

The references cited herein, including both those cited below and above, are incorporated herein by reference.

Bazzicalupo P. 1983. Caenorhabditis elegans: A model system for the study of nematodes. In: “Molecular Biology of Parasites,” (J. Guardiola, L. Luzzatto, W. Trager, Eds.) Raven Press, New York, pp. 73-92.

Bloomquist J R. 2003. Chloride channels as tools for developing selective insecticides. Arch. Insect Biochem. Physiol. 54: 145-156.

Cabantchik Z I and Greger R. 1992. Chemical probes for anion transporters of mammalian cell membranes. Am. J. Physiol. 262(4 Pt 1): C803-27.

Chen G, Webster J, Li J, Hu K, Zhu J. 2001. Anti-inflammatory and psoriasis treatment and protein kinase inhibition by hydroxystilbenes and novel stilbene derivatives and analogs. Patent No. WO 2001042231.

Hu K, Li J, Webster, J. 2000. Antibiotic production in relation to bacterial growth and nematode development in Photorhabdus-Heterorhabditis infected Galleria mellonella larvae. FEMS Microbiol. Lett. 189: 219-223.

Hu K, Li J, Webster J. 1999. Nematicidal metabolites produced by Photorhabdus luminescens (Enterobacteriaceae), bacterial symbiont of entomopathogenic nematodes. Nematology 1: 457-469.

Hu K, Li J, Webster J. 1997. Quantitative analysis of a bacteria-derived antibiotic in nematode-infected insects using HPLC-UV and TLC-UV methods. J. Chromatog. B 703: 177-183.

1. A method of treating a multicellular host organism attacked, bitten, infected, or parasitized by at least one nematode, at least one insect, at least one acarine, or combinations of two or all three of these, said method comprising contacting the host organism or its environment with at least one compound in amount sufficient to disrupt the activity of at least one anion transporter of the nematode, insect, or acarine, the disruption reducing the viability or activity of the nematode, insect, or acarine.

2. The method of claim 1, wherein the multicellular organism is a plant.

3. The method of claim 2, wherein the plant is a food crop.

4. The method of claim 1, wherein the multicellular organism is an animal.

5. The method of claim 4, wherein the animal is a mammal.

6. The method of claim 5, wherein the mammal is a farm animal.

7. The method of claim 1, wherein the compound is a stilbene compound.

8. A method of protecting a multicellular host organism attacked, bitten, infected, or parasitized by at least one nematode, insect, or acarine, said method comprising contacting the nematode or insect or acarine with at least one compound in amount sufficient to disrupt the activity of at least one anion transporter of the nematode, insect, or acarine, the disruption reducing the activity or viability of the nematode or insect or acarine.

9. The method of claim 8, wherein the multicellular organism is a plant.

10. The method of claim 9, wherein the plant is a food crop.

11. The method of claim 8, wherein the multicellular organism is an animal.

12. The method of claim 11, wherein the animal is a mammal.

13. The method of claim 12, wherein the mammal is a farm animal.

14. The method of claim 8, wherein the compound is a stilbene compound.

15. A method of identifying a test compound having nematicidal, insecticidal, or acaricidal activity, said method comprising:
exposing at least one anion transporter from at least one nematode, insect, or Acari to at least one test compound,
providing sufficient time for the anion transporter(s) and test compound(s) to come into contact; and
determining whether one or more of the test compounds affected the activity of one or more of the anion transporters,
wherein an effect on the activity of at least one anion transporter indicates nematicidal or insecticidal or acaricidal activity of at least one of the test compounds.

16. The method of claim 15, wherein the method is practiced, at least partially, in vitro.

17. The method of claim 15, wherein at least one of the test compounds is a stilbene compound.

18. The method of claim 15, wherein determining comprises measuring the activity of at least on anion transporter that was exposed to at least one test compound.

19. The method of claim 15, further comprising determining the activity of the anion transporters under the same conditions, but without the presence of the test compound(s).

20. The method of claim 15, further comprising identifying the test compound(s) showing an effect on the anion transporter(s).

21. The method of claim 15, further comprising repeating the exposing, providing, and determining steps more than one time,
wherein each iteration of the steps is performed using fewer test compound(s), fewer AT, or fewer of both.

22. The method of claim 15, comprising determining the effect of a single test compound on a single AT.

23. The method of claim 15, further comprising correlating the activity of the test compound(s) on inhibition of growth or killing of at least one nematode, insect, or acarine.
